(19) United States
(12) Patent Application Publication
(10) Pub. No.: US 2023/0019048 A1
(43) Pub. Date: Jan. 19, 2023

(54) Targeted Temperature Management Systems, Pads, and Methods Thereof

(71) Applicant: C. R. Bard, Inc., Franklin Lakes, NJ (US)

(72) Inventors: Madeline Stich, Thornton, CO (US); Sean E. Walker, Platteville, CO (US); Gabriel A. Johnston, Broomfield, CO (US)

(21) Appl. No.: 17/857,997

(22) Filed: Jul. 5, 2022

Related U.S. Application Data
(60) Provisional application No. 63/223,500, filed on Jul. 19, 2021.

(51) Int. Cl. A61F 7/02; A61F 7/00

(52) U.S. Cl. CPC A61F 7/02; A61F 7/007; A61F 7/0085; A61F 2007/0054; A61F 2007/0093; A61F 2007/0247; A61F 2007/0253

(57) ABSTRACT

Disclosed are systems, pads, and methods for targeted temperature management. A pad can include a multilayered pad body, a pad inlet connector, and a pad outlet connector. The pad body can include a plurality of separable longitudinal portions, wherein each longitudinal portion of the longitudinal portions includes a conduit, a patient-interfacing layer over the conduit, and an insulation layer over the conduit opposite the patient-interfacing layer. The conduit can be configured to convey a temperature-controlled fluid as a supply fluid from a hydraulic system or convey a return fluid back to the hydraulic system. The patient-interfacing layer can include a thermally conductive medium configured for placement on a patient’s body. The insulation layer can include an insulative foam. The pad inlet connector can be configured for charging the pad with the supply fluid while the pad outlet connector can be configured for discharging the return fluid from the pad.

### PRIORITY

This application claims the benefit of priority to U.S. Provisional Pat. Application No. 63/223,500, filed Jul. 19, 2021, which is incorporated by reference in its entirety into this application.

### BACKGROUND

Targeted temperature management (“TTM”) is a treatment for maintaining therapeutic body temperatures (e.g., hypothermia, hyperthermia, etc.) in patients to improve their outcomes in different medical situations. Current systems for TTM generally use pads placed on different portions of the patient’s bodies for circulating temperature-controlled fluid (e.g., cooled fluid or warmed fluid) about the patients to induce or maintain therapeutic body temperatures. Being that the pads must maintain sufficient contact with the patient’s bodies for optimum thermal conductivity between the temperature-controlled fluid and the patient’s bodies, improvements to such pads continues to be an active area of research and development.

Disclosed herein are TTM systems, pads, and methods thereof.

### SUMMARY

Disclosed herein is a pad for TTM including, in some embodiments, a multilayered pad body, a pad inlet connector, and a pad outlet connector. The pad body includes a plurality of separable longitudinal portions. Each longitudinal portion of the plurality of longitudinal portions includes a conduit, a patient-interfacing layer over the conduit, and an insulation layer over the conduit opposite the patient-interfacing layer. The conduit is configured to convey a temperature-controlled fluid as a supply fluid from a hydraulic system or convey a return fluid back to the hydraulic system. The patient-interfacing layer includes a thermally conductive medium configured for placement on a patient’s body. The insulation layer includes an insulative foam. The pad inlet connector is configured for charging one or more conduits of the plurality of longitudinal portions of the pad body with the supply fluid. The pad outlet connector configured for discharging the return fluid from the one-or-more conduits, one or more other conduits of the plurality of longitudinal portions of the pad body than the one-or-more conduits, or a combination thereof.

In some embodiments, the pad inlet connector includes an inlet manifold, and the pad outlet connector includes an outlet manifold. The inlet manifold is configured for the charging of the one-or-more conduits with the supply fluid. The outlet manifold is configured for the discharging of the return fluid from the one-or-more conduits, the one-or-more other conduits, or the combination thereof.

In some embodiments, each conduit of the one-or-more conduits is in parallel with other conduits of the one-or-more conduits.

In some embodiments, each conduit of the one-or-more conduits and the one-or-more other conduits is fluidly connected to another conduit of the one-or-more conduits and the one-or-more other conduits. Such fluid connections provide one or more fluid paths through the pad from the pad inlet connector to the pad outlet connector.

In some embodiments, the fluid connections between the one-or-more conduits and the one-or-more other conduits are between ends of the one-or-more conduits and the one-or-more other conduits, internal portions of the one-or-more conduits and the one-or-more other conduits, or a combination thereof. The plurality of longitudinal portions are separable between the fluid connections.

In some embodiments, the insulation layer is perforated between each longitudinal portion of the plurality of longitudinal portions. Such perforations allow the plurality of longitudinal portions to be separated and selectively placed upon the patient’s body.

Also disclosed herein is a pad for TTM including, in some embodiments, a multilayered pad body, a pad inlet connector, and a pad outlet connector. The pad body includes a conduit layer, a patient-interfacing layer over the conduit layer, and an insulation layer over the conduit layer opposite the patient-interfacing layer. The conduit layer includes a plurality of conduits moveably disposed in a thermally conductive medium. Each conduit of the plurality of conduits is configured to convey a temperature-controlled fluid as a supply fluid from a hydraulic system or convey a return fluid back to the hydraulic system. The patient-interfacing layer is configured for placement on a patient’s body. The patient-interfacing layer includes a thermally conductive film configured to retain the plurality of conduits and the thermally conductive medium in the conduit layer. The pad inlet connector is configured for charging the conduit layer with the supply fluid. The pad outlet connector is configured for discharging the return fluid from the conduit layer.

In some embodiments, one or more conduits of the plurality of conduits are configured to be moved away from one or more other conduits of the plurality of conduits for selective placement of the one-or-more conduits, the one-or-more other conduits, or a combination thereof upon the patient’s body.

In some embodiments, the pad inlet connector includes an inlet manifold, and the pad outlet connector includes an outlet manifold. The inlet manifold is configured for charging the plurality of conduits with the supply fluid. The outlet manifold is configured for discharging the return fluid from the plurality of conduits.

In some embodiments, each conduit of the plurality of conduits is in parallel with other conduits of the plurality of conduits.

In some embodiments, each conduit of the plurality of conduits is fluidly connected to another conduit of the plurality of conduits. Such fluid connections provide one or more fluid paths through the pad from the pad inlet connector to the pad outlet connector.

In some embodiments, the fluid connections between the plurality of conduits are between ends of the plurality of conduits, internal portions of the plurality of conduits, or a combination thereof. The plurality of conduits are moveable between the fluid connections.

In some embodiments, the pad further includes a plurality of thermoelectric devices and an electrical connector configured for establishing an operable connection with a control module for controlling the plurality of thermoelectric devices. The plurality of thermoelectric devices are disposed in the conduit layer orthogonal to the plurality of conduits. The plurality of thermoelectric devices are configured to undergo a temperature change upon application of a voltage across the plurality of thermoelectric devices.

Also disclosed herein is a pad for TTM including, in some embodiments, a multilayered pad body, a pad inlet connector, and a pad outlet connector. The pad body includes a conduit layer and a patient-interfacing layer over the conduit layer. The conduit layer includes a plurality of conduits randomly formed throughout an open-cell foam. The plurality of conduits are configured to convey a temperature-controlled fluid as a supply fluid from a hydraulic system and convey a return fluid back to the hydraulic system. The patient-interfacing layer includes a thermally conductive medium configured for placement on a patient’s body. The pad inlet connector is configured for charging the conduit layer with the supply fluid. The pad outlet connector is configured for discharging the return fluid from the conduit layer.

In some embodiments, the patient-interfacing layer further includes a thermally conductive film over the thermally conductive medium. The thermally conductive film is configured to retain the thermally conductive medium between the conduit layer and the thermally conductive film.

In some embodiments, the pad inlet connector includes an inlet manifold, and the pad outlet connector includes an outlet manifold. The inlet manifold is configured for charging one or more inlet portions of the conduit layer with the supply fluid. The outlet manifold is configured for discharging the return fluid from one or more outlet portions of the conduit layer.

In some embodiments, fluid connections between conduits of the plurality of conduits are between ends of the conduits, internal portions of the conduits, or a combination thereof, thereby providing one or more fluid paths through the pad from the pad inlet connector to the pad outlet connector.

In some embodiments, the pad is configured to wrap around a portion of the patient’s body using one or more straps or cuffs.

Also disclosed herein is a pad for TTM including, in some embodiments, a multilayered pad body, a plurality of pad inlet connectors, and a plurality of pad outlet connectors. The pad body includes a conduit layer and a thermally conductive medium over the conduit layer in a plurality of separable sections of the pad body. The conduit layer includes a plurality of conduits configured to convey a temperature-controlled fluid as a supply fluid from a hydraulic system and convey a return fluid back to the hydraulic system. The thermally conductive medium is configured for placement on a patient’s body. The plurality of pad inlet connectors correspond in number to the plurality of separable sections of the pad body or greater in number than the plurality of separable sections. Each pad inlet connector of the plurality of pad inlet connectors is configured for charging the conduit layer of its corresponding separable section with the supply fluid. The plurality of pad outlet connectors correspond in number to the plurality of separable sections of the pad body or greater in number than the plurality of separable sections. Each pad outlet connector of the plurality of pad outlet connectors is configured for discharging the return fluid from the conduit layer of its corresponding separable section.

In some embodiments, the plurality of pad inlet connectors includes a primary pad inlet connector, and the plurality of pad outlet connectors includes a primary pad outlet connector. The primary pad inlet connector and the primary pad outlet connector correspond to any separable section of the plurality of separable sections.

In some embodiments, the plurality of pad inlet connectors includes one or more secondary pad inlet connectors, and the plurality of pad outlet connectors includes one or more secondary pad outlet connectors. The one-or-more secondary pad inlet connectors and the one-or-more secondary pad outlet connectors correspond to a primary separable section of the plurality of separable sections. The one-or-more secondary pad inlet connectors are configured to fluidly connect to the one-or-more primary pad outlet connectors respectively of one or more secondary separable sections of the plurality of separable sections. The one-or-more secondary pad outlet connectors are configured to fluidly connect to the one-or-more primary pad inlet connectors respectively of the one-or-more secondary separable sections such that each secondary separable section of the one-or-more secondary separable sections is fluidly connected to the primary separable section of the plurality of separable sections in parallel to other secondary separable sections of the one-or-more secondary separable sections before any secondary separable section is separated from the primary separable section.

In some embodiments, the plurality of separable sections are perforated between the separable sections. Such perforation allows the plurality of separable sections to be separated and selectively placed upon the patient’s body.

In some embodiments, the pad body further includes an impermeable film between the conduit layer and the thermally conductive medium. The impermeable film is configured to retain the supply fluid in the conduit layer.

In some embodiments, the conduit layer includes a plurality of protrusions extending from the conduit layer toward the impermeable film. The plurality of protrusions are configured to promote even flow of the temperature-controlled fluid.

In some embodiments, the thermally conductive medium includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

In some embodiments, the pad further includes a release liner over the thermally conductive medium in a ready-to-use state of the pad. The release liner is configured to maintain integrity of at least the thermally conductive medium prior to use of the pad.

Also disclosed herein is a pad for TTM including, in some embodiments, a plurality of interlinked pad sections of the pad. Each pad section of the plurality of interlinked pad sections includes a multilayered pad body, a plurality of loops formed by way of a plurality of through holes in the pad body, one or more pad inlet connectors, and one or more pad outlet connectors. The pad body includes a conduit layer and a thermally conductive medium over the conduit layer. The conduit layer includes one or more conduits configured to convey a temperature-controlled fluid as a supply fluid from a hydraulic system or convey a return fluid back to the hydraulic system. The thermally conductive medium is configured for placement on a patient’s body. Each loop of the plurality of loops is interlinked with a loop of one or more other pad sections of the plurality of interlinked pad sections. The one-or-more pad inlet connectors are configured for charging the conduit layer with the supply fluid. The one-or-more pad outlet connectors are configured for discharging the return fluid from the conduit layer to a pad inlet connector of another pad section of the plurality of interlinked pad sections or the hydraulic system.

In some embodiments, each pad section of the plurality of interlinked pad sections includes three loops for the plurality of loops.

In some embodiments, each pad section of the plurality of interlinked pad sections includes four loops for the plurality of loops.

In some embodiments, each pad section of the plurality of interlinked pad sections includes at least on pad inlet connectors for the one-or-more pad inlet connectors and two pad outlet connectors for the one-or-more pad outlet connectors.

In some embodiments, the pad body further includes an impermeable film between the conduit layer and the thermally conductive medium. The impermeable film is configured to retain the supply fluid in the conduit layer.

In some embodiments, the conduit layer includes a plurality of protrusions extending from the conduit layer toward the impermeable film. The plurality of protrusions is configured to promote even flow of the temperature-controlled fluid.

In some embodiments, the thermally conductive medium includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

In some embodiments, the pad further comprises a release liner over the thermally conductive medium in a ready-to-use state of the pad. The release liner is configured to maintain integrity of at least the thermally conductive medium prior to use of the pad.

Also disclosed herein is a pad for TTM including, in some embodiments, a multilayered pad body, a plurality of thermally conductive extension arms extending from the pad body, a pad inlet connector, and a pad outlet connector. The pad body includes a conduit layer and a thermally conductive medium over the conduit layer. The conduit layer includes one or more conduits configured to convey a temperature-controlled fluid as a supply fluid from a hydraulic system and convey a return fluid back to the hydraulic system. The thermally conductive medium is configured for placement on a patient’s body. Each arm of the plurality of extension arms is configured to conform to the patient’s body and thermally transfer heat between the patient’s body and the pad body. The pad inlet connector is configured for charging the conduit layer with the supply fluid. The pad outlet connector is configured for discharging the return fluid from the conduit layer to the hydraulic system.

In some embodiments, the pad further includes webbing between adjacent arms of the plurality of extension arms. The webbing includes an insulative foam and the thermally conductive medium over the insulative foam. Again, the thermally conductive medium is configured for placement on the patient’s body.

In some embodiments, the pad body further includes an impermeable film between the conduit layer and the thermally conductive medium. The impermeable film is configured to retain the supply fluid in the conduit layer.

In some embodiments, the conduit layer includes a plurality of protrusions extending from the conduit layer toward the impermeable film. The plurality of protrusions is configured to promote even flow of the temperature-controlled fluid.

In some embodiments, the thermally conductive medium includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

In some embodiments, the pad further includes a release liner over the thermally conductive medium in a ready-to-use state of the pad. The release liner is configured to maintain integrity of at least the thermally conductive medium prior to use of the pad.

Also disclosed herein is a pad for TTM including, in some embodiments, a multilayered pad body, a plurality of thermally conductive extension arms extending from the pad body, an electrical connector. The pad body includes an insulative layer, a plurality of thermoelectric devices disposed in the insulative layer, and a thermally conductive medium over the insulative layer. The plurality of thermoelectric devices are configured to undergo a temperature change upon application of a voltage across the plurality of thermoelectric devices. The thermally conductive medium is configured for placement on a patient’s body. Each arm of the plurality of extension arms is configured to conform to the patient’s body and thermally transfer heat between the patient’s body and the pad body. The electrical connector configured for establishing an operable connection with a control module for controlling the plurality of thermoelectric devices.

In some embodiments, the pad further includes webbing between adjacent arms of the plurality of extension arms. The webbing includes the insulative foam and the thermally conductive medium over the insulative foam. Again, the thermally conductive medium is configured for placement on the patient’s body.

Also disclosed herein is a pad for TTM including, in some embodiments, a multilayered pad body, a plurality of extension arms extending from the pad body, a pad inlet connector, and a pad outlet connector. The pad body includes a conduit layer and a thermally conductive medium over the conduit layer. The conduit layer includes one or more conduits configured to convey a temperature-controlled fluid as a supply fluid from a hydraulic system or convey a return fluid back to the hydraulic system. The thermally conductive medium is configured for placement on a patient’s body. Each arm of the plurality of extension arms is an extension of the pad body including both the conduit layer and the thermally conductive medium. Each arm of the plurality of extension arms is configured to stretch or contract as needed for conforming to the patient’s body. The pad inlet connector is configured for charging the conduit layer with the supply fluid. The pad outlet connector is configured for discharging the return fluid from the conduit layer to the hydraulic system.

In some embodiments, the plurality of extension arms including a braided mesh reinforcement layer lining the one-or conduits of the conduit layer. The braided mesh reinforcement is configured to prevent the one-or-more conduits from collapsing when any arm of the plurality of extension arms is stretched.

In some embodiments, the pad body further includes one or more through holes. The one-or-more through holes are configured for passing one or more medical device therethrough, checking skin integrity of skin therethrough, or both when the pad is disposed on the patient’s body.

In some embodiments, the pad body further includes an impermeable film between the conduit layer and the thermally conductive medium. The impermeable film is configured to retain the supply fluid in the conduit layer.

In some embodiments, the conduit layer includes a plurality of protrusions extending from the conduit layer toward the impermeable film. The plurality of protrusions are configured to promote even flow of the temperature-controlled fluid.

In some embodiments, the thermally conductive medium includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

In some embodiments, the thermally conductive medium over the plurality of extension arms includes one or more tackifiers to increase a tack of the thermally conductive medium over the plurality of extension arms.

In some embodiments, the pad further comprises a release liner over the thermally conductive medium in a ready-to-use state of the pad. The release liner is configured to maintain integrity of at least the thermally conductive medium prior to use of the pad.

Also disclosed herein is a system, for TTM including, in some embodiments, a control module, a primary fluid delivery line (“FDL”), and one or more pads configured for placement on one or more portions of a patient’s body, respectively. The control module includes a hydraulic system configured to provide a temperature-controlled fluid. The primary FDL is configured to convey the temperature-controlled fluid from the hydraulic system as a supply fluid and convey the temperature-controlled fluid back to the hydraulic system as a return fluid. Each pad of the one-or-more pads selected from a paragraph of paragraphs [0004]-[0043] and [0046]-[0053].

In some embodiments, each pad of the one-or-more pads includes a multilayered pad body, a pad inlet connector, and a pad outlet connector. The pad body includes a conduit layer, a patient-interfacing layer over the conduit layer, and an insulation layer over the conduit layer opposite the patient-interfacing layer. The conduit layer includes a plurality of conduits moveably disposed in a thermally conductive medium. Each conduit of the plurality of conduits is configured to convey the temperature-controlled fluid as the supply fluid from the hydraulic system or convey the return fluid back to the hydraulic system. The patient-interfacing layer is configured for placement on the patient’s body. The patient-interfacing layer includes a thermally conductive film configured to retain the plurality of conduits and the thermally conductive medium in the conduit layer. The pad inlet connector is configured for charging the conduit layer with the supply fluid. The pad outlet connector is configured for discharging the return fluid from the conduit layer.

In some embodiments, one or more conduits of the plurality of conduits are configured to be moved away from one or more other conduits of the plurality of conduits for selective placement of the one-or-more conduits, the one-or-more other conduits, or a combination thereof upon the patient’s body.

In some embodiments, the pad inlet connector includes an inlet manifold, and the pad outlet connector includes an outlet manifold. The inlet manifold is configured for charging the plurality of conduits with the supply fluid. The outlet manifold is configured for discharging the return fluid from the plurality of conduits.

In some embodiments, each conduit of the plurality of conduits is in parallel with other conduits of the plurality of conduits.

In some embodiments, each conduit of the plurality of conduits is fluidly connected to another conduit of the plurality of conduits. Such fluid connections provide one or more fluid paths through the pad from the pad inlet connector to the pad outlet connector.

In some embodiments, fluid connections between the plurality of conduits are between ends of the plurality of conduits, internal portions of the plurality of conduits, or a combination thereof. The plurality of conduits are moveable between the fluid connections.

In some embodiments, the pad body further includes a plurality of thermoelectric devices and an electrical connector configured for establishing an operable connection with the control module for controlling the plurality of thermoelectric devices. The plurality of thermoelectric devices are disposed in the conduit layer orthogonal to the plurality of conduits. The plurality of thermoelectric devices are configured to undergo a temperature change upon application of a voltage across the plurality of thermoelectric devices.

In some embodiments, the control module further includes a console including one or more processors, primary memory including read-only memory (“ROM”) and random-access memory (“RAM”), and instructions stored in the ROM. The instructions are configured to instantiate one or more processes in the RAM for TTM with the control module.

In some embodiments, the hydraulic system includes a chiller evaporator, a heater, a hydraulic-system outlet, and a hydraulic-system inlet. The chiller evaporator is configured for fluid cooling. The heater is configured for fluid heating. The chiller evaporator and the heater, together, are configured to provide the temperature-controlled fluid. The hydraulic-system outlet is configured for discharging the supply fluid from the hydraulic system. The hydraulic-system inlet is configured for charging the hydraulic system with the return fluid to continue to produce the temperature-controlled fluid.

In some embodiments, the system further includes one or more secondary FDLs. The one or more secondary FDLs are configured to convey the supply fluid from the hydraulic system by way of the primary FDL and convey the return fluid back to the hydraulic system by way of the primary FDL.

Also disclosed herein is a system for TTM including, in some embodiments, a control module, a primary cable, and one or more pads. The control module includes one or more processors, primary memory including ROM and RAM, and instructions stored in the ROM. The instructions are configured to instantiate one or more processes in the RAM for TTM with the control module. The primary cable is configured to enable application of a voltage across one or more thermoelectric devices such that the one-or-more thermoelectric devices undergo a temperature change. The one-or-more pads are configured for placement on one or more portions of a patient’s body, respectively. Each pad of the one-or-more pads includes the one-or more thermoelectric devices as set forth for the pad of either paragraph [0044] or [0045].

Also disclosed herein is a method for TTM including, in some embodiments, a pad-placing step, a pad-charging step, and a fluid-circulating step. The pad-placing step includes placing a pad on a patient’s body. The pad is selected from a paragraph of paragraphs [0004]-[0043] and [0046]-[0053]. The pad-charging step includes charging one or more conduits of a conduit layer of the pad with a supply fluid. The supply fluid is provided by a hydraulic system of a control module by way of a combination of fluidly connected FDLs including a primary FDL and a secondary FDL. The fluid-circulating step includes circulating the supply fluid through the conduit layer to cool or warm the patient’s body as needed in accordance with TTM.

In some embodiments, the pad includes a multilayered pad body including a pad inlet connector, and a pad outlet connector. The pad body includes the conduit layer, a patient-interfacing layer over the conduit layer, and an insulation layer over the conduit layer opposite the patient-interfacing layer. The conduit layer includes a plurality of conduits moveably disposed in a thermally conductive medium. Each conduit of the plurality of conduits is configured to convey a temperature-controlled fluid as the supply fluid from the hydraulic system of the control module or convey a return fluid back to the hydraulic system. The patient-interfacing layer is configured for placement on the patient’s body. The patient-interfacing layer includes a thermally conductive film configured to retain the plurality of conduits and the thermally conductive medium in the conduit layer. The pad inlet connector is configured for charging the conduit layer with the supply fluid. The pad outlet connector is configured for discharging the return fluid from the conduit layer.

In some embodiments, one or more conduits of the plurality of conduits are configured to be moved away from one or more other conduits of the plurality of conduits for selective placement of the one-or-more conduits, the one-or-more other conduits, or a combination thereof upon the patient’s body.

In some embodiments, the pad inlet connector includes an inlet manifold, and the pad outlet connector includes an outlet manifold. The inlet manifold is configured for charging the plurality of conduits with the supply fluid. The outlet manifold is configured for discharging the return fluid from the plurality of conduits.

In some embodiments, each conduit of the plurality of conduits is in parallel with other conduits of the plurality of conduits.

In some embodiments, each conduit of the plurality of conduits is fluidly connected to another conduit of the plurality of conduits. Such fluid connections provide one or more fluid paths through the pad from the pad inlet connector to the pad outlet connector.

In some embodiments, fluid connections between the plurality of conduits are between ends of the plurality of conduits, internal portions of the plurality of conduits, or a combination thereof. The plurality of conduits are moveable between the fluid connections.

In some embodiments, the pad body further includes a plurality of thermoelectric devices and an electrical connector configured for establishing an operable connection with the control module for controlling the plurality of thermoelectric devices. The plurality of thermoelectric devices are disposed in the conduit layer orthogonal to the plurality of conduits. The plurality of thermoelectric devices are configured to undergo a temperature change upon application of a voltage across the plurality of thermoelectric devices.

In some embodiments, the method further includes a first secondary FDL-connecting step and a second secondary FDL-connecting step. The first secondary FDL-connecting step includes fluidly connecting a secondary FDL outlet connector at a pad-connecting end of the secondary FDL to a pad inlet connector. The second secondary FDL-connecting step includes fluidly connecting a secondary FDL inlet connector at the pad-connecting end of the secondary FDL to a pad outlet connector.

In some embodiments, the fluid-circulating step transfers heat between the temperature-controlled fluid and the patient’s body by thermal conduction through the conduit layer.

In some embodiments, the fluid-circulating step includes circulating a cool fluid through the conduit layer to bring the patient into hypothermia from normothermia.

In some embodiments, the fluid-circulating step includes circulating a warm fluid through the conduit layer to bring the patient into normothermia from hypothermia.

In some embodiments, the fluid-circulating step includes circulating a warm fluid through the conduit layer to bring the patient into hyperthermia from normothermia.

In some embodiments, the fluid-circulating step includes circulating a cool fluid through the conduit layer to bring the patient into normothermia from hyperthermia.

These and other features of the concepts provided herein will become more apparent to those of skill in the art in view of the accompanying drawings and following description, which describe particular embodiments of such concepts in greater detail.

### DRAWINGS

FIG. 1 illustrates a system for TTM including a control module and one or more pads in accordance with some embodiments.

FIG. 2 illustrates left and right torso pads of the one-or-more pads in accordance with some embodiments.

FIG. 3 illustrates left and right thigh pads of the one-or-more pads in accordance with some embodiments.

FIG. 4 illustrates a hydraulic pad having a pad body with a plurality of separable longitudinal portions in accordance with some embodiments.

FIG. 5 illustrates the hydraulic pad with the pad body separated along the longitudinal portions in accordance with some embodiments.

FIG. 6 illustrates a cross section of the pad body of the one-or-more hydraulic pads of FIGS. 4 and 5 in accordance with some embodiments.

FIG. 7 illustrates a hydraulic or hybrid pad having a pad body with a plurality of movable conduits disposed in a thermally conductive medium in accordance with some embodiments.

FIG. 8 illustrates a hydraulic pad having a pad body with a plurality of conduits randomly formed throughout an open-cell foam in accordance with some embodiments.

FIG. 9 illustrates a hydraulic pad having a pad body with a plurality of separable sections in accordance with some embodiments.

FIG. 10 illustrates a detailed view of the hydraulic pad of FIG. 9 along an interface of the separable sections in accordance with some embodiments.

FIG. 11 illustrates a multilayered pad body of the pad of at least FIG. 10 in accordance with some embodiments.

FIG. 12 illustrates a hydraulic pad having a pad body with a plurality of interlinked pad sections in accordance with some embodiments.

FIG. 13 illustrates a hydraulic or thermoelectric pad having a pad body with a plurality of thermally conductive extension arms extending from the pad body in accordance with some embodiments.

FIG. 14 illustrates a hydraulic pad having a pad body with a plurality of deformable extension arms extending from the pad body in accordance with some embodiments.

FIG. 15 illustrates a hydraulic system of the control module in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Unless defined otherwise, all technical and scientific terms used herein have the same meaning as commonly understood by those of ordinary skill in the art.

As set forth above, TTM is a treatment for maintaining therapeutic body temperatures (e.g., hypothermia, hyperthermia, etc.) in patients to improve their outcomes in different medical situations. Current systems for TTM generally use pads placed on different portions of the patient’s bodies for circulating temperature-controlled fluid (e.g., cooled fluid or warmed fluid) about the patients to induce or maintain therapeutic body temperatures. Being that the pads must maintain sufficient contact with the patient’s bodies for optimum thermal conductivity between the temperature-controlled fluid and the patient’s bodies, improvements to such pads continues to be an active area of research and development.

Disclosed herein are TTM systems, pads, and methods thereof.

### Systems for TTM

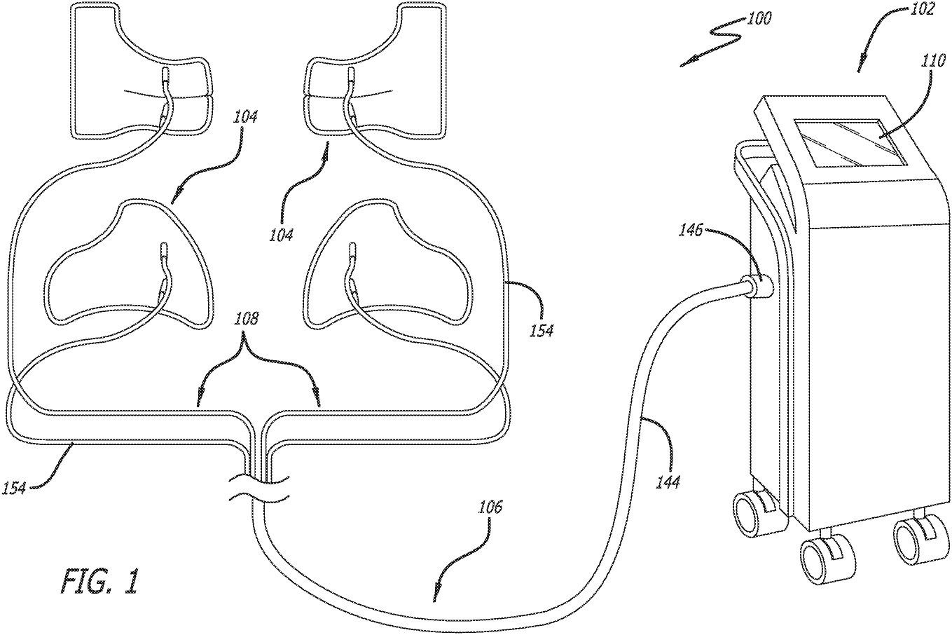

FIG. 1 illustrates a system 100 for TTM including a control module 102 and one or more pads 104 in accordance with some embodiments.

As shown, the system 100 can include the control module 102 configured for hydraulic TTM, the one-or-more pads 104 such as any of the hydraulic or hybrid pads 204, 304, 404, 504, 604, 704, and 804 set forth below, a primary FDL 106, and one or more secondary FDLs 108 corresponding in number to the one-or-more pads 104. Description for the control module 102 configured for hydraulic TTM is set forth immediately below. Description for the one-or-more pads 104 including the hydraulic or hybrid pads 204, 304, 404, 504, 604, 704, and 804 and the one-or-more secondary FDLs 108 is set forth in the following section.

The control module 102 can include a console 110 with an integrated display screen configured as a touchscreen for operating the control module 102. The control module 102 can include one or more processors, primary memory including ROM and RAM, and instructions stored in the ROM. The instructions are configured to instantiate one or more processes in the RAM for hydraulic TTM with the control module 102 when executed by the one-or-more processors.

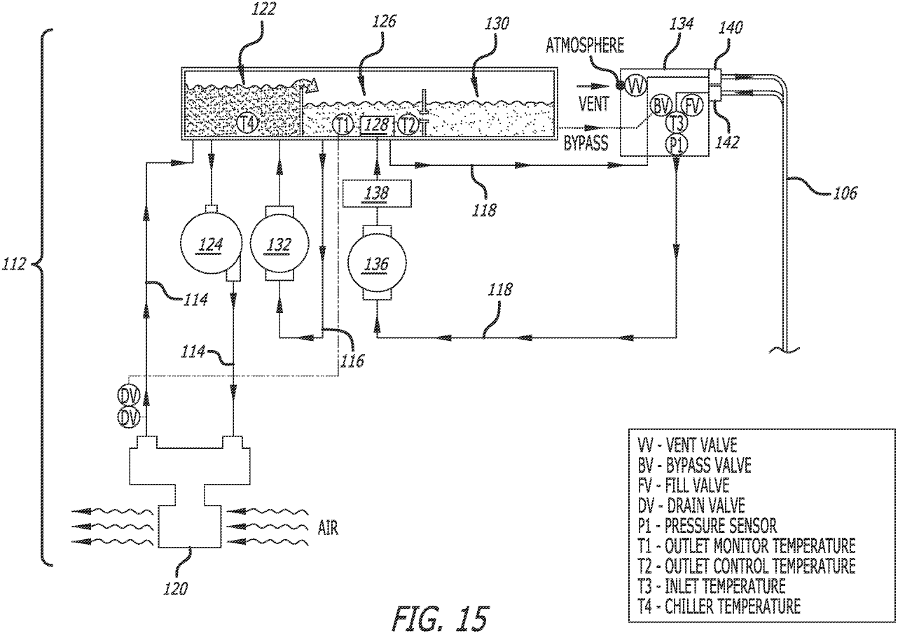

FIG. 15 illustrates a hydraulic system 112 of the control module 102 in accordance with some embodiments.

The control module 102 can also include the hydraulic system 112, which can include a chiller circuit 114, a mixing circuit 116, and a circulating circuit 118 for providing a temperature-controlled fluid.

The chiller circuit 114 can be configured for cooling a fluid (e.g., water, ethylene glycol, a combination of water and ethylene glycol, etc.) to produce a cooled fluid, which cooled fluid, in turn, can be for mixing with the mixed fluid in the mixing tank 126 set forth below to produce a supply fluid for TTM. The chiller circuit 114 can include a chiller evaporator 120 configured for the cooling of the fluid passing therethrough. The fluid for the cooling by the chiller evaporator 120 is provided by a chiller tank 122 using a chiller pump 124 of the chiller circuit 114.

The mixing circuit 116 can be configured for mixing spillover of the cooled fluid from the chiller tank 122 with a mixed fluid in a mixing tank 126 of the mixing circuit 116. The mixing circuit 116 can include a heater 128 in the mixing tank 126 configured for heating the mixed fluid to produce a heated fluid, which can be mixed with the cooled fluid in any ratio to provide a supply tank 130 of the circulating circuit 118 with the supply fluid of a desired temperature for TTM. Indeed, the chiller evaporator 120 and the heater 128, together, are configured to cooperate to provide the temperature-controlled fluid. The mixing circuit 116 can include a mixing pump 132 configured to pump the fluid from the mixing tank 126 into the chiller tank 122 for producing the cooled fluid as well as the spillover of the cooled fluid for the mixing tank 126.

The circulating circuit 118 can be configured for circulating the supply fluid for TTM, which includes circulating the supply fluid provided by a manifold 134 through the one-or-more pads 104 using a circulation pump 136 directly or indirectly governed by a flow meter 138 of the circulating circuit 118. The manifold 134 can include an outlet 140 configured for discharging the supply fluid (e.g., a cooled fluid or a warmed fluid as indicated) from the hydraulic system 112 and an inlet 142 configured for charging the hydraulic system 112 with return fluid from the one-or-more pads 104 to continue to produce the supply fluid.

The primary FDL 106 can include primary tubing 144 configured with a supply lumen to convey the supply fluid from the hydraulic system 112 by way of a lumen of the primary tubing 144 when fluidly connected to the hydraulic system 112. Likewise, the primary tubing 144 is configured with a return lumen to convey the return fluid back to the hydraulic system 112 by way of the lumen of the primary tubing 144 when fluidly connected to the hydraulic system 112.

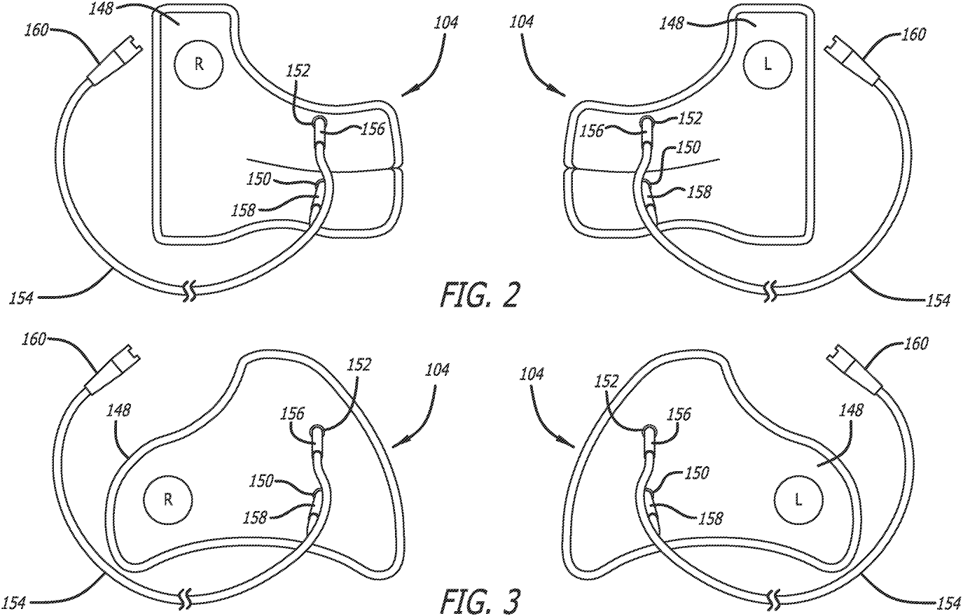

The primary FDL 106 can include a pair of opposing connector ends, wherein each connector end of the pair of connector ends includes a corresponding primary FDL connector. For example, a connector end of the pair of connector ends can include a control module-connecting primary FDL connector 146 configured to fluidly connect to a control-module connector including both the outlet 140 and the inlet 142 of the hydraulic system 112. Another connector end of the pair of connector ends can include a secondary FDL-connecting primary FDL connector (not shown) configured to fluidly connect to the primary FDL-connecting secondary FDL connector 160 set forth below. (See FIGS. 2 and 3.) The secondary FDL-connecting primary FDL connector can include both an outlet and an inlet.

The system 100 for TTM can additionally or alternatively include the control module 102 configured for thermoelectric TTM, the one-or-more pads 104 such as any of the thermoelectric or hybrid pads 304 and 704 set forth below, a primary cable, and, optionally, one or more secondary cables corresponding in number to the one-or-more pads 104. Description for the control module 102 additionally or alternatively configured for thermoelectric TTM is set forth immediately below. Description for the one-or-more pads 104 including the thermoelectric or hybrid pads 304 and 704 is set forth in the following section.

Again, the control module 102 can include the console 110 with the integrated display screen configured as the touchscreen for operating the control module 102. The control module 102 can include the one-or-more processors, the primary memory including the ROM and the RAM, and the instructions stored in the ROM. However, the instructions are additionally or alternatively configured to instantiate the one-or-more processes in the RAM for thermoelectric TTM with the control module 102 when executed by the one-or-more processors.

The primary cable is configured to enable the control module to apply a voltage across one or more thermoelectric devices such that the one-or-more thermoelectric devices undergo a temperature change. Each pad of the thermoelectric or hybrid pads 304 and 704 includes the one-or more thermoelectric devices as set forth below.

### Pads for TTM

Before description of the hydraulic pads 204, 304, 404, 504, 604, 704, and 804, the thermoelectric pad 704, or the hybrid pad 304, some general features of at least the hydraulic pads and hybrid pads 204, 304, 404, 504, 604, 704, and 804 are provided by way the description of the one-or-more pads 104 set forth immediately below. Thus, it should be understood that the one-or-more pads 104 are generic with respect to at least the hydraulic and hybrid pads 204, 304, 404, 504, 604, 704, and 804. If not wholly generic to the foregoing pads, the description of the one-or-more pads 104 set forth immediately below provides at least some general features of the hydraulic and hybrid pads 204, 304, 404, 504, 604, 704, and 804.

FIGS. 2 and 3 illustrate left and right pads of the one-or-more pads 104 respectively for a torso and thighs of a patient in accordance with some embodiments.

As shown, each pad of the one-or-more pads 104 can include a multilayered pad body 148, a pad inlet connector 150, and a pad outlet connector 152.

The pad body 148 can vary in construction as set forth below with respect to the hydraulic and hybrid pads 204, 304, 404, 504, 604, 704, and 804. That said, each pad of at least the hydraulic and hybrid pads 504, 604, 704, and 804 can include a conduit layer or the like configured to convey the temperature-controlled fluid as the supply fluid from the hydraulic system or convey the return fluid back to the hydraulic system.

The pad body 148 can also vary in shape. Indeed, while FIGS. 2 and 3 illustrate left and right pads of the one-or-more pads 104 respectively for a patient’s torso and thighs, the one-or-more pads 104 can be configured for placement on any one or more portions of the patient’s body, respectively, not just the torso or thighs.

The pad inlet connector 150 can include a pad inlet configured for charging the conduit layer or the like with the supply fluid, while the pad outlet connector 152 can include a pad outlet configured for discharging the return fluid from the conduit layer.

A pad of the one-or-more pads 104 can include a secondary FDL of the one-or-more secondary FDLs 108. For example, the secondary FDL can be pre-connected to the pad as packaged. Alternatively, the secondary FDL can be provided in a same or different package as the pad but not connected to the pad.

The secondary FDL can include secondary tubing 154 configured with a supply lumen to convey the supply fluid from the primary FDL 106 when the primary FDL 106 is connected to the hydraulic system 112 of the control module 102. Likewise, the secondary tubing 154 can be configured with a return lumen to convey the return fluid back to the primary FDL 106 when the primary FDL 106 is connected to the hydraulic system 112 of the control module 102.

The secondary FDL can be split at a pad-connecting end of the secondary FDL. The pad-connecting end of the secondary FDL can include a pair of pad-connecting secondary FDL connectors. A pad-connecting secondary FDL outlet connector 156 of the pair of pad-connecting secondary FDL connectors can be configured to fluidly connect to the pad inlet connector 150. A pad-connecting secondary FDL inlet connector 158 of the pair of pad-connecting secondary FDL connectors can be configured to fluidly connect to the pad outlet connector 152.

The secondary FDL need not be split at a primary FDL-connecting end of the secondary FDL like the pad-connecting end of the secondary FDL. Indeed, an unsplit primary FDL-connecting end of the secondary FDL facilitates quickly connecting the one-or-more secondary FDLs 108 to the primary FDL 106. Accordingly, the primary FDL-connecting end of the secondary FDL can include a single primary FDL-connecting secondary FDL connector 160 configured to fluidly connect to the secondary FDL-connecting primary FDL connector (not shown) set forth above. The primary FDL-connecting secondary FDL connector 160 can include both an inlet and an outlet corresponding to the outlet and the inlet of the secondary FDL-connecting primary FDL connector.

Any pad of the hydraulic or hybrid pads 204, 304, 404, 504, 604, 704, and 804 can include a secondary FDL of the one-or-more secondary FDLs 108 as set forth above. Notably, the hydraulic pad 504 can include additional FDLs to the one-or-more secondary FDLs 108 as set forth below. In addition, the hydraulic pad 604 can include additional or alternative FDLs to the one-or-more secondary FDLs 108 as set forth below.

Analogous to a pad of the one-or-more pads 104 including a secondary FDL of the one-or-more secondary FDLs 108, the thermoelectric and hybrid pads 304 and 704 can include a secondary cable of the one-or-more cables. For example, the secondary cable can be pre-connected to the pad as packaged. Alternatively, the secondary cable can be provided in a same or different package as the pad but not connected to the pad.

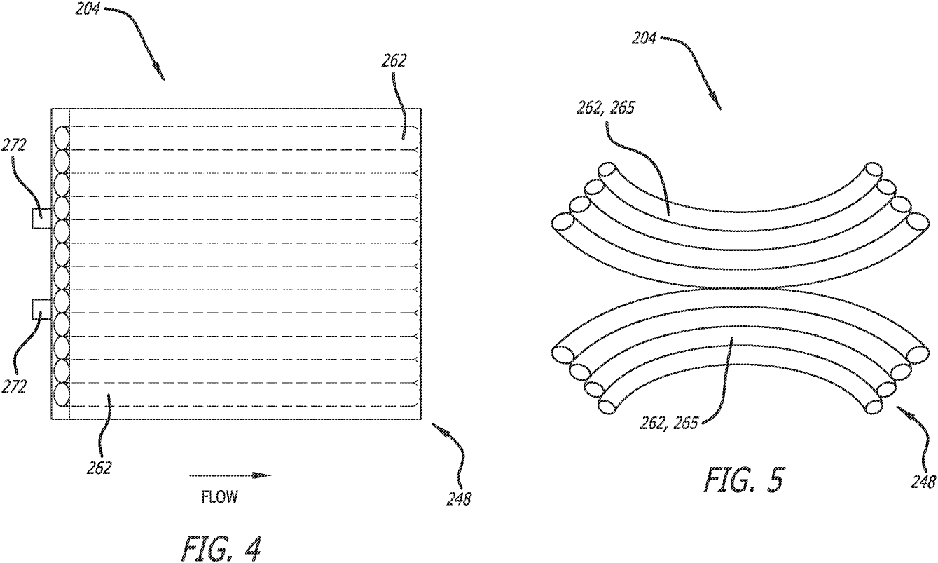

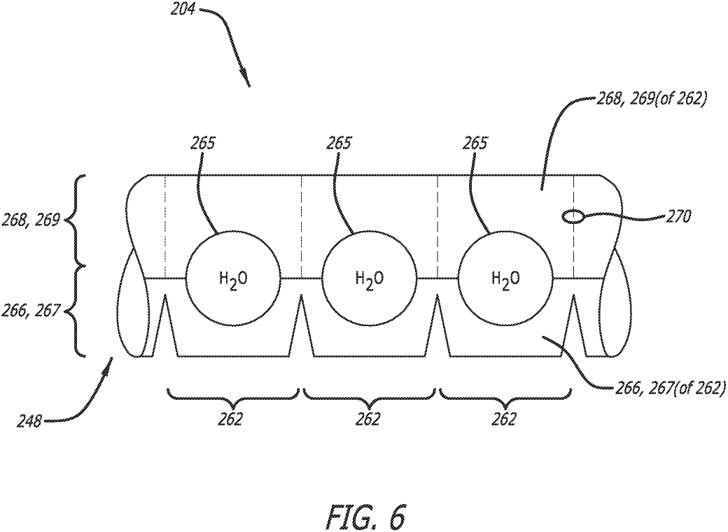

FIG. 4 illustrates a hydraulic pad 204 having a pad body 248 with a plurality of separable longitudinal portions 262 in accordance with some embodiments. FIG. 5 illustrates the pad 204 with the pad body 248 separated along the longitudinal portions 262 in accordance with some embodiments. FIG. 6 illustrates a cross section of the pad body 248 of the hydraulic pad 204 of in accordance with some embodiments.

As shown, the pad 204 includes the pad body 248, which can be a multilayered pad body as shown in FIG. 6. While not shown, the pad 204 can include a pad inlet connector and a pad outlet connector such as the pad inlet connector 150 and the pad outlet connector 152.

Again, the pad body 248 includes the separable longitudinal portions 262. Each longitudinal portion of the longitudinal portions 262 includes a conduit 265 for a plurality of conduits 265, a patient-interfacing layer 266 over the conduits 265, and an insulation layer 268 over the conduits 265 opposite the patient-interfacing layer 266.

Each conduit of the conduits 265 is configured to convey the temperature-controlled fluid as the supply fluid from the hydraulic system 112 or convey the return fluid back to the hydraulic system 112. Indeed, each conduit of the conduits 265 is fluidly connected to another conduit of the conduits 265 to convey the supply fluid from the hydraulic system 112 or convey the return fluid back to the hydraulic system 112. Such fluid connections provide one or more fluid paths through the pad 204 from the pad inlet connector 150 to the pad outlet connector 152. For example, the fluid connections between the conduits 265 are between ends of the conduits 265 (e.g., through the inlet manifold and outlet manifolds) such that the conduits 265 are fluidly connected in parallel with other conduits of the conduits 265; however, some internal portions of the conduits 265 can be fluidly connected such that those portions of the conduits 265 are fluidly connected in series. Notably, the longitudinal portions 262 are separable between the fluid connections.

The patient-interfacing layer 266 includes a thermally conductive medium 267 configured for placement on a patient’s body. The thermally conductive medium 267 includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

The insulation layer 268 includes an insulative foam 269. The insulation layer 268 including the insulative foam 269 is perforated between each longitudinal portion of the longitudinal portions 262 with perforations 270. Such perforations allow the longitudinal portions 262 to be separated, for example, by tabs 272, and selectively placed upon the patient’s body. Notably, a stretchable filament or bundle of filaments (e.g., twisted or braided filaments) can be used between the longitudinal portions 262 to hold the pad 204 together.

The pad inlet connector 150 is configured for charging one or more conduits of the conduits 265 with the supply fluid. The pad outlet connector 152 is configured for discharging the return fluid from the one-or more conduits or one or more other conduits of the conduits 265. The pad inlet connector 150 can include an inlet manifold and the pad outlet connector 152 can likewise include an outlet manifold. The inlet manifold is configured for the charging of the conduits 265 with the supply fluid. The outlet manifold is configured for the discharging of the return fluid from the conduits 265. With such manifolds, the conduits 265 are easily fluidly connected in parallel.

FIG. 7 illustrates a hydraulic or hybrid pad 304 having a pad body 348 with a plurality of movable conduits 365 disposed in the thermally conductive medium 267 in accordance with some embodiments.

As shown, the pad 304 includes the pad body 348, which can be a multilayered pad body. While not shown, the pad 304 can include a pad inlet connector and a pad outlet connector such as the pad inlet connector 150 and the pad outlet connector 152.

The pad body 348 includes a conduit layer 364, a patient-interfacing layer 366 over the conduit layer 364, and an insulation layer 368 over the conduit layer 364 opposite the patient-interfacing layer 366.

The conduit layer 364 includes the conduits 365 moveably disposed in the thermally conductive medium 267. Indeed, one or more conduits of the conduits 365 are configured to be moved away or separated from one or more other conduits of the conduits 365 for selective placement of the one-or-more conduits, the one-or-more other conduits, or a combination thereof upon the patient’s body. In other words, a first set of the conduits 365 are configured to be moved away or separated from a second set of the conduits 365 for selective placement of the first set of the conduits 365, the second set of the conduits 365, or a combination thereof upon the patient’s body.

Each conduit of the conduits 365 is configured to convey the temperature-controlled fluid as the supply fluid from the hydraulic system 112 or convey the return fluid back to the hydraulic system 112. Indeed, each conduit of the conduits 365 is fluidly connected to another conduit of the conduits 365 to convey the supply fluid from the hydraulic system 112 or convey the return fluid back to the hydraulic system 112. Such fluid connections provide one or more fluid paths through the pad 304 from the pad inlet connector 150 to the pad outlet connector 152. For example, the fluid connections between the conduits 365 are between ends of the conduits 365 (e.g., through the inlet manifold and outlet manifolds) such that the conduits 365 are fluidly connected in parallel with other conduits of the conduits 365; however, some internal portions of the conduits 365 can be fluidly connected such that those portions of the conduits 365 are fluidly connected in series. Notably, the conduits 365 are moveable between the fluid connections.

The patient-interfacing layer 366 is configured for placement on the patient’s body. The patient-interfacing layer 366 includes a thermally conductive film 367 configured to retain the conduits 365 and the thermally conductive medium 267 in the conduit layer 364. Again, the thermally conductive medium 267 includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

The pad inlet connector 150 is configured for charging the conduit layer 364 with the supply fluid. The pad outlet connector 152 is configured for discharging the return fluid from the conduit layer 364. The pad inlet connector 150 can include an inlet manifold and the pad outlet connector 152 can likewise include an outlet manifold. The inlet manifold is configured for the charging the conduits 365 with the supply fluid. The outlet manifold is configured for discharging the return fluid from the conduits 365. With such manifolds, the conduits 365 are easily fluidly connected in parallel.

The pad 304 can further include a plurality of thermoelectric devices 374 and an electrical connector configured for establishing an operable connection with the control module 102 for controlling the thermoelectric devices 374. The thermoelectric devices 374 are disposed in the conduit layer 364 orthogonal to the conduits 365. The thermoelectric devices 374 are configured to undergo a temperature change upon application of a voltage across the thermoelectric devices 374.

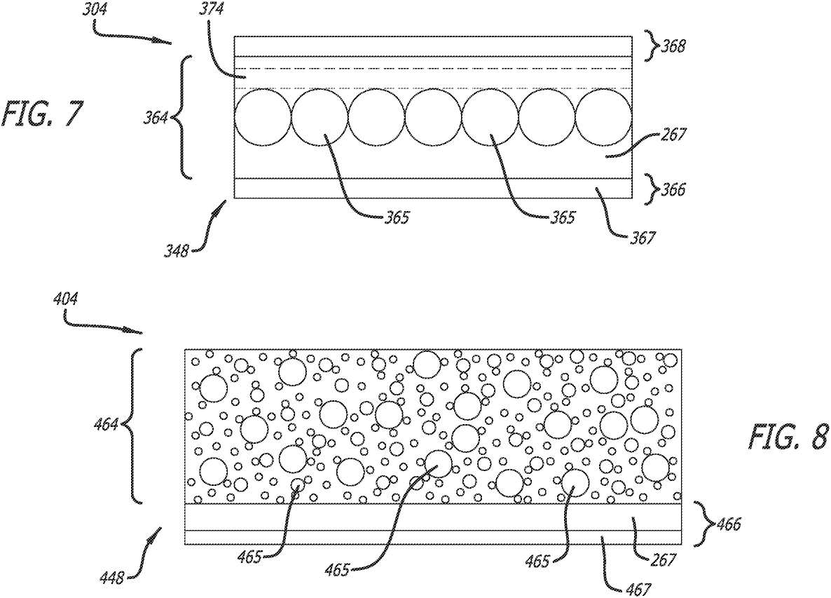

FIG. 8 illustrates a hydraulic pad 404 having a pad body 448 with a plurality of conduits 465 randomly formed throughout an open-cell foam in accordance with some embodiments.

As shown, the pad 404 includes the pad body 448, which can be a multilayered pad body. While not shown, the pad 404 can include a pad inlet connector and a pad outlet connector such as the pad inlet connector 150 and the pad outlet connector 152.

The pad body 448 includes a conduit layer 464 of the open-cell foam and a patient-interfacing layer 466 over the conduit layer. Optionally, the conduit layer 464 opposite the patient-interfacing layer 466 is sealed with a sealant or a film.

The conduit layer 464 includes the conduits 465 randomly formed throughout the open-cell foam. Each conduit of the conduits 465 is configured to convey the temperature-controlled fluid as the supply fluid from the hydraulic system 112 and convey the return fluid back to the hydraulic system 112. Indeed, each conduit of the conduits 465 is fluidly connected to another conduit of the conduits 465 to convey the supply fluid from the hydraulic system 112 and convey the return fluid back to the hydraulic system 112. Such fluid connections provide one or more fluid paths through the pad 404 from the pad inlet connector 150 to the pad outlet connector 152. For example, the fluid connections between the conduits 465 are between ends of the conduits 465, internal portions of the conduits 465, or a combination thereof, thereby providing the one-or-more fluid paths through the pad 404 from the pad inlet connector 150 to the pad outlet connector 152.

The patient-interfacing layer 466 includes the thermally conductive medium 267 and a thermally conductive film 467 over the thermally conductive medium 267 configured for placement on the patient’s body. Again, the thermally conductive medium 267 includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels. The thermally conductive film 467 is configured to retain the thermally conductive medium 267 between the conduit layer 464 and the thermally conductive film 467.

The pad inlet connector 150 is configured for charging the conduit layer 464 with the supply fluid. The pad outlet connector 152 is configured for discharging the return fluid from the conduit layer 464. The pad inlet connector 150 can include an inlet manifold and the pad outlet connector 152 can likewise include an outlet manifold. The inlet manifold is configured for the charging one or more inlet portions of the conduit layer 464 with the supply fluid. The outlet manifold is configured for discharging the return fluid from one or more outlet portions of the conduit layer 464.

The pad 404 is configured to wrap around a portion of the patient’s body using one or more straps or cuffs. The one-or-more straps or cuffs can include rolling or moving pressure applicators configured to roll or move over the pad body 448 to push the temperature-controlled fluid through the conduit layer 464.

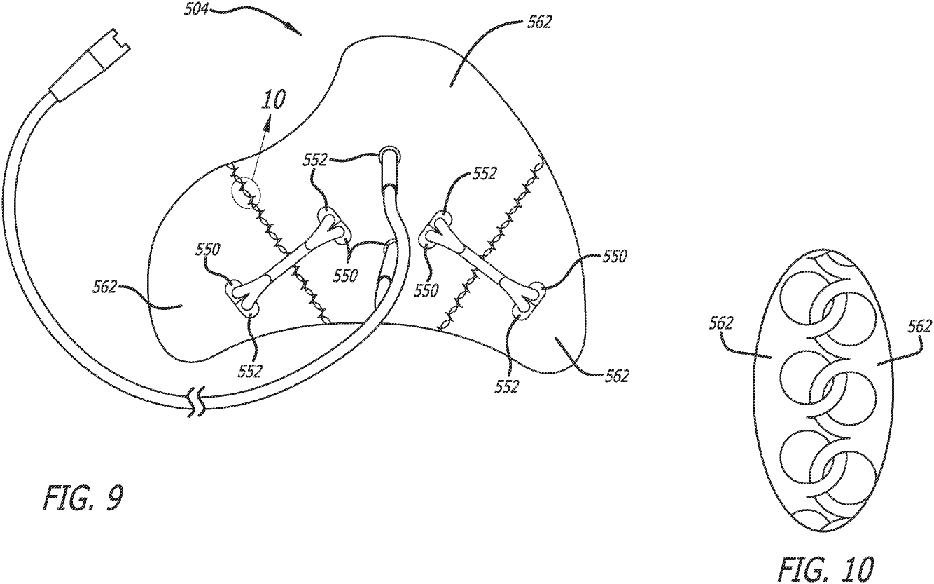

FIG. 9 illustrates a hydraulic pad 504 having a pad body 548 with a plurality of separable sections 562 in accordance with some embodiments. FIG. 10 illustrates a detailed view of the hydraulic pad 504 of FIG. 9 along an interface of the separable sections 562 in accordance with some embodiments.

As shown, the pad 504 includes the pad body 548, which can be a multilayered pad body. The pad 504 can include a plurality of pad inlet connectors 550 and a plurality of pad outlet connectors 552.

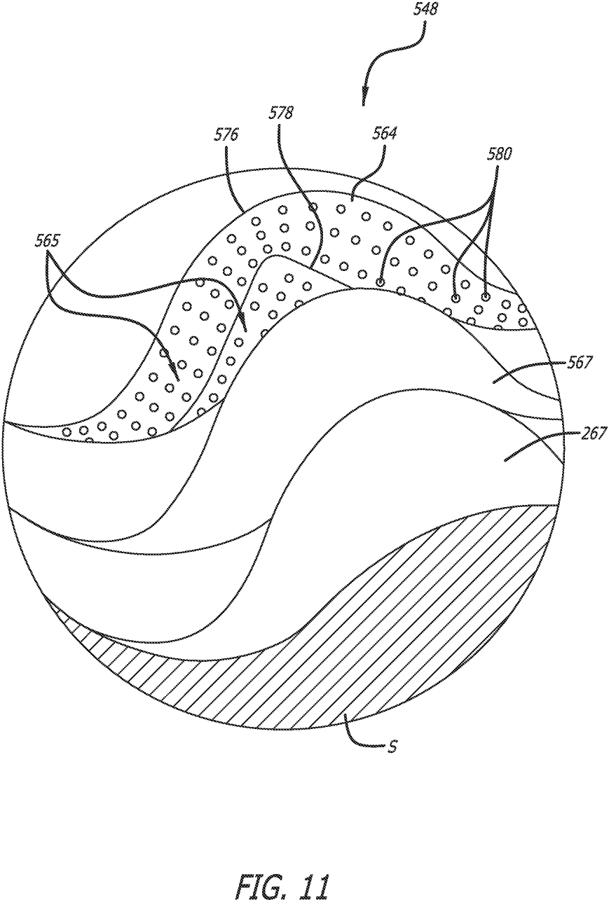

FIG. 11 illustrates the pad body 548 of the pad 504 in accordance with some embodiments.

Again, the pad body 548 includes the separable sections 562. The separable sections 562 are perforated or interlinked as shown in FIG. 10 between the separable sections 562. Such perforation or interlinks allows the separable sections 562 to be separated and selectively placed upon the patient’s body. Notably, each separable section of the separable sections 562 has a same body construction. Indeed, the pad body 548 includes a conduit layer 564, an impermeable film 567 over the conduit layer 564, and the thermally conductive medium 267 over the impermeable film 567 in the separable sections 562 of the pad body 548.

The conduit layer 564 includes a perimetrical wall 576 and one or more inner walls 578 extending from the conduit layer 564 toward the impermeable film 567. Together with the impermeable film 567, the perimetrical wall 576 and the one-or-more inner walls 578 form one or more conduits 565 configured to convey through the conduit layer 564 the temperature-controlled fluid as the supply fluid from the hydraulic system 112 or the return fluid back to the hydraulic system 112. The conduit layer can also include a plurality of protrusions 580 extending from the conduit layer 564 toward the impermeable film 567. The protrusions 580 are configured to promote even flow of the temperature-controlled fluid as the supply fluid or the return fluid when conveyed through the conduit layer 564.

The impermeable film 567 between the conduit layer 564 and the thermally conductive medium 267 is configured to retain the temperature-controlled fluid as the supply fluid or the return fluid in the conduit layer 564 when the conveyed through the conduit layer 564. In addition, the impermeable film 567 is configured to allow efficient energy transfer between the conduit layer 564 and the thermally conductive medium 267.

The thermally conductive medium 267 is configured for placement on skin S (see FIG. 11) of a portion (e.g., torso, thigh, etc.) of the patient’s body for thermal conduction through the thermally conductive medium 267. Again, the thermally conductive medium 267 includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels. Such a thermally conductive medium is configured to conformably adhere to the portion of the patient’s body for optimum thermal conduction but without irritation.

While not shown, the pad 504 can include a release liner over the thermally conductive medium 267 in a ready-to-use state of the pad 504. Such a release liner, which maintains an integrity of the thermally conductive medium 267 prior to use of the pad 504, is configured to expose the thermally conductive medium 267 when the release liner is removed from the pad 504.

The pad inlet connectors 550 correspond in number to the separable sections 562 of the pad body 548 or are greater in number than the separable sections 562. Each pad inlet connector of the pad inlet connectors 550 is configured for charging the conduit layer 564 of its corresponding separable section with the supply fluid. The pad outlet connectors 552 also correspond in number to the separable sections 562 of the pad body or are greater in number than the separable sections 562. Each pad outlet connector of the pad outlet connectors 552 is configured for discharging the return fluid from the conduit layer 564 of its corresponding separable section.

The pad inlet connectors 550 include a primary pad inlet connector and the pad outlet connectors 552 include a primary pad outlet connector. The primary pad inlet connector and the primary pad outlet connector correspond to any separable section of the separable sections 562. For example, the primary pad inlet connector and the primary pad outlet connector of a primary or central separable section of the separable sections 562 of the pad 504 respectively correspond to the pad inlet connector 150 and the pad outlet connector 152 identified in FIG. 3.

The pad inlet connectors 550 include one or more secondary pad inlet connectors and the pad outlet connectors 552 include one or more secondary pad outlet connectors. For example, FIG. 9 illustrates two secondary pad inlet connectors and two secondary pad outlet connectors that correspond to the primary separable section of the separable sections 562. (Notably, the two secondary pad inlet connectors of the primary separable section of the separable sections 562 are those that do not correspond to the pad inlet connector 150 identified in FIG. 3. Likewise, the two secondary pad outlet connectors of the primary separable section of the separable sections 562 are those that do not correspond to the pad outlet connector 152 identified in FIG. 3.) The two secondary pad inlet connectors of the primary separable section of the separable sections 562 are configured to fluidly connect to the primary pad outlet connectors respectively of the secondary separable sections of the separable sections 562 shown in FIG. 9, wherein the secondary separable sections of the separable sections 562 flank the primary separable section. The two secondary pad outlet connectors of the primary separable section of the separable sections 562 are configured to fluidly connect to the primary pad inlet connectors respectively of the secondary separable sections of the separable sections 562 shown in FIG. 9 such that each secondary separable section of the separable sections 562 is fluidly connected to the primary separable section of the separable sections 562 in parallel before any secondary separable section is separated from the primary separable section.

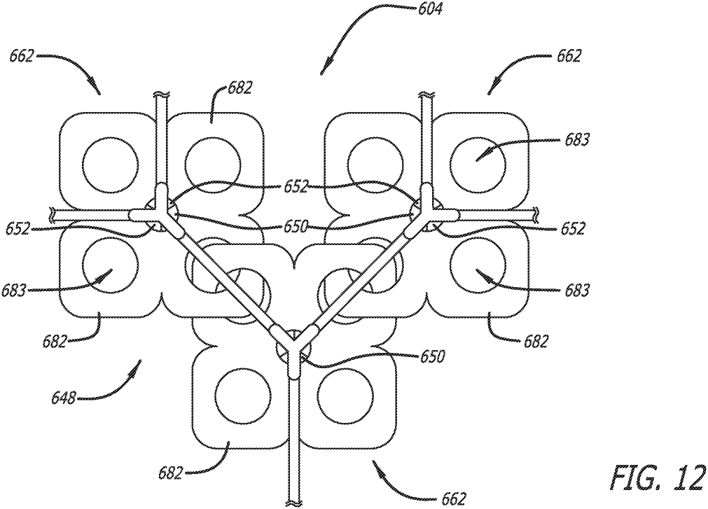

FIG. 12 illustrates a hydraulic pad 604 having a pad body 648 with a plurality of interlinked pad sections 662 in accordance with some embodiments.

As shown, the pad 604 includes the interlinked pad sections 662, in which each pad section of the interlinked pad sections 662 forms a portion of the pad body 648 including a plurality of loops 682 formed by way of a plurality of through holes 683 in each pad section of the interlinked pad sections 662, one or more pad inlet connectors 650, and one or more pad outlet connectors 652.

Again, the pad 604 includes the interlinked pad sections 662, which are interlinked as shown in FIG. 12 between the loops 682 and through holes 683 of each pad section of the interlinked pad sections 662. Each loop of the loops 682 is interlinked with a loop of one or more other pad sections of the interlinked pad sections 662. Notably, while each pad section of the interlinked pad sections 662 is shown with four loops, other configurations are possible such that each pad section of the interlinked pad sections 662 has, for example, three or five loops. Interlinks allow the interlinked pad sections 662 to be conformably placed upon the patient’s body. In addition, space between each pad section of the interlinked pad sections 662 is useful for checking skin integrity therethrough when the pad 604 is disposed on the patient’s body.

Each pad section of the interlinked pad sections 662 has a same body construction. As set forth above for each separable section of the separable sections 562 of the pad body 548 of the pad 504, the body construction each pad section of the interlinked pad sections 662 can include the conduit layer 564, the impermeable film 567 over the conduit layer 564, and the thermally conductive medium 267 over the impermeable film 567 configured for placement on a patient’s body. The conduit layer 564 includes the one-or-more conduits configured to convey the temperature-controlled fluid as the supply fluid from the hydraulic system 112 and convey the return fluid back to the hydraulic system 112.

While not shown, the pad 604 can include a release liner over the thermally conductive medium 267 in a ready-to-use state of the pad 604. Such a release liner, which maintains an integrity of the thermally conductive medium 267 prior to use of the pad 604, is configured to expose the thermally conductive medium 267 when the release liner is removed from the pad 604.

The one-or-more pad inlet connectors 650 are configured for charging the conduit layer 564 of a pad section of the interlinked pad sections 662 with the supply fluid. The one-or-more pad outlet connectors 652 are configured for discharging the return fluid from the conduit layer 564 of the foregoing pad section of the interlinked pad sections 662 to a pad inlet connector of another pad section of the interlinked pad sections 662 or the hydraulic system 112. As shown, each pad section of the interlinked pad sections 662 includes at least one pad inlet connector for the one-or-more pad inlet connectors 650 and two pad outlet connectors for the one-or-more pad outlet connectors 652.

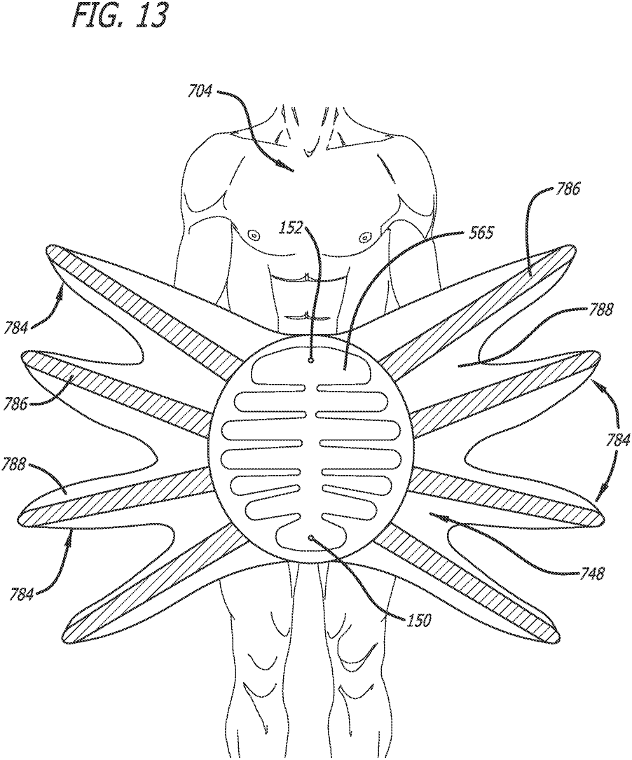

FIG. 13 illustrates a hydraulic or thermoelectric pad 704 having a pad body 748 with a plurality of thermally conductive extension arms 784 extending from the pad body 748 in accordance with some embodiments.

As shown, the pad 704 includes the pad body 748, which can be a multilayered pad body, the extension arms 784 extending from the pad body 748, a pad inlet connector such as the pad inlet connector 150, and a pad outlet connector such as the pad outlet connector 152 when the pad 704 is a hydraulic pad. When the 704 is a thermoelectric pad, the pad 704 includes an electrical connector instead of the pad inlet connector 150 and the pad outlet connector 152.

As set forth for the pad body 548 of the pad 504, when the pad 704 is a hydraulic pad, the pad body 748 can include the conduit layer 564, the impermeable film 567 over the conduit layer 564, and the thermally conductive medium 267 over the impermeable film 567 configured for placement on a patient’s body. The conduit layer 564 includes the one-or-more conduits 565 configured to convey the temperature-controlled fluid as the supply fluid from the hydraulic system 112 and convey the return fluid back to the hydraulic system 112. When the pad 704 is a thermoelectric pad, the pad body 748 can include an insulative layer, a plurality of thermoelectric devices disposed in the insulative layer, and the thermally conductive medium over the insulative layer. The thermoelectric devices are configured to undergo a temperature change upon application of a voltage across the one-or-more thermoelectric devices.

While not shown, the pad 704 can include a release liner over the thermally conductive medium 267 in a ready-to-use state of the pad 704. Such a release liner, which maintains an integrity of the thermally conductive medium 267 prior to use of the pad 704, is configured to expose the thermally conductive medium 267 when the release liner is removed from the pad 704.

Each arm of the extension arms 784 is configured to conform to the patient’s body and thermally transfer heat between the patient’s body and the pad body 748 by way of thermal conduction along the extension arm. Such extension arms can include metal inserts 786 with a high thermal conductivity. To further facilitate thermal conduction along the extension arms 784, the pad 704 can further include webbing 788 between adjacent arms of the extension arms 784. The webbing 788 includes the insulative foam and the thermally conductive medium 267 over the insulative foam such that the thermally conductive medium 267 is placed on the patient’s body.

The pad inlet connector 150 is configured for charging the conduit layer 564 with the supply fluid. The pad outlet connector 152 is configured for discharging the return fluid from the conduit layer 564 to the hydraulic system 112. The alternative electrical connector is configured for establishing an operable connection with the control module 102 for controlling the thermoelectric devices.

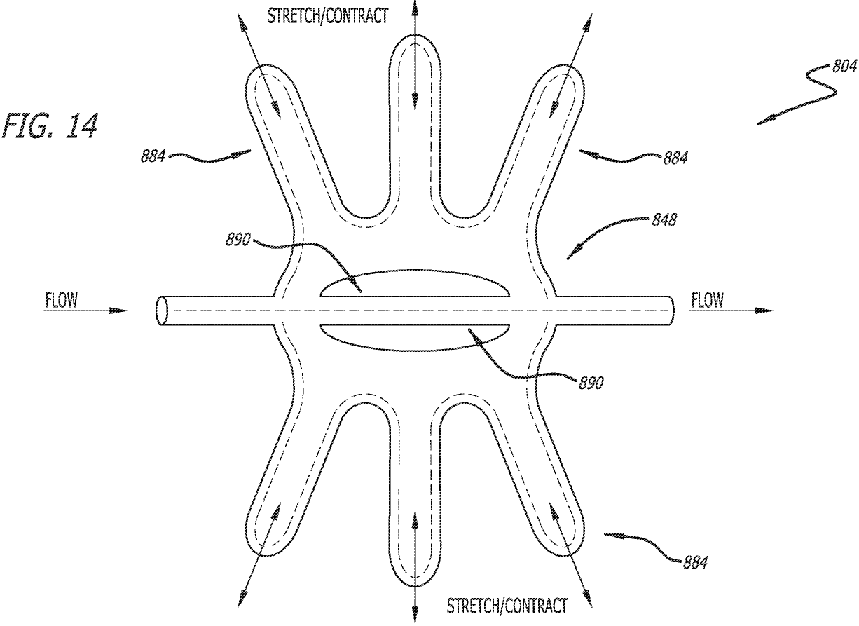

FIG. 14 illustrates a hydraulic pad 804 having a pad body 848 with a plurality of deformable extension arms 884 extending from the pad body 848 in accordance with some embodiments.

As shown, the pad 804 includes the pad body 848, which can be a multilayered pad body, and the plurality of extension arms 884 extending from the pad body 848. While not shown, the pad 804 can include a pad inlet connector such as the pad inlet connector 150 and a pad outlet connector such as the pad outlet connector 152.

As set forth for the pad body 548 of the pad 504, the pad body 848 can include the conduit layer 564, the impermeable film 567 over the conduit layer 564, and the thermally conductive medium 267 over the impermeable film 567 configured for placement on a patient’s body. The conduit layer 564 includes the one-or-more conduits 565 configured to convey the temperature-controlled fluid as the supply fluid from the hydraulic system 112 and convey the return fluid back to the hydraulic system 112. Notably, the pad body 848 further includes one or more through holes 890. The one-or-more through holes 890 are configured for passing one or more medical devices therethrough, checking skin integrity of the patient’s skin therethrough, or both when the pad 804 is disposed on the patient’s body.

As set forth above for the pad body 548 of the pad 504, the thermally conductive medium 267 includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels. However, the thermally conductive medium 367 over the extension arms 884 can include one or more tackifiers to increase a tack of the thermally conductive medium 267 over the extension arms 884.

While not shown, the pad 804 can include a release liner over the thermally conductive medium 267 in a ready-to-use state of the pad 804. Such a release liner, which maintains an integrity of the thermally conductive medium 267 prior to use of the pad 804, is configured to expose the thermally conductive medium 267 when the release liner is removed from the pad 804.

Each arm of the extension arms 884 is an extension of the pad body 848 including both the conduit layer 564 and the thermally conductive medium 267 optionally including the one-or-more tackifiers. Each arm of the extension arms 884 is configured to deform such as stretch or contract without permanent deformation as needed for conforming to the patient’s body. The extension arms 884 can include a braided mesh reinforcement layer lining the one-or-more conduits 565 of the conduit layer 564. The braided mesh reinforcement is configured to prevent the one-or-more conduits 565 from collapsing when any arm of the extension arms 884 is deformed (e.g., stretched).

The pad inlet connector is configured for charging the conduit layer with the supply fluid. The pad outlet connector is configured for discharging the return fluid from the conduit layer to the hydraulic system 112.

### Methods

Methods of the system 100 or the one-or-more pads 104 include methods of use. For example, a method of using the system 100 for TTM can include one or more steps selected from a release liner-removing step, a pad-placing step, an FDL-connecting step, a pad-charging step, and a fluid-circulating step.

As set forth above, a pad of the one-or-more pads 104 can include the release liner over the thermally conductive medium 267. In such embodiments, the method can include the release liner-removing step. The release liner-removing step includes removing the release liner to expose the thermally conductive medium 267.

The pad-placing step can include placing the pad on a patient’s body in contact with skin S of the patient’s body. (See, for example, FIG. 11.) Depending upon the pad of the one-or-more pads 104 set forth above, the portion of the pad placed in contact with the skin S of the patient’s body includes the thermally conductive medium 267.

If a secondary FDL is not pre-connected to the pad, the method can include the FDL-connecting step. The FDL-connecting step, in turn, can include a first secondary FDL-connecting step and a second secondary FDL-connecting step. The first secondary FDL-connecting step includes fluidly connecting a secondary FDL outlet connector at a pad-connecting end of the secondary FDL to a pad inlet connector such as the pad inlet connector 150. The second secondary FDL-connecting step includes fluidly connecting a secondary FDL inlet connector at the pad-connecting end of the secondary FDL to a pad outlet connector such as the pad outlet connector 152.

The pad-charging step includes charging the one-or-more conduits 565 of the conduit layer 564 of the pad with the supply fluid of the temperature-controlled fluid. As set forth above, the supply fluid is provided by the hydraulic system 112 of the control module 102 by way of a combination of fluidly connected FDLs including the primary FDL 106 and the secondary FDL 108.

The fluid-circulating step can include circulating the supply fluid through the conduit layer 564 of the pad to cool or warm the patient’s body as needed in accordance with TTM. Indeed, the fluid-circulating step can include transferring heat between the temperature-controlled fluid and the patient’s body by thermal conduction through the conduit layer 564. For example, the fluid-circulating step can include circulating a cool fluid through the conduit layer 564 to bring the patient into hypothermia from normothermia. In another example, the fluid-circulating step can include circulating a warm fluid through the conduit layer 564 to bring the patient into normothermia from hypothermia. In yet another example, the fluid-circulating step can include circulating a warm fluid through the conduit layer 564 to bring the patient into hyperthermia from normothermia. In yet another example, the fluid-circulating step can include circulating a cool fluid through the conduit layer 564 to bring the patient into normothermia from hyperthermia.

While the method set forth above is described with reference to a single pad of the one-or-more pads 104, it should be understood that any number of pads of the one-or-more pads 104 can be used as necessary to effectuate a desired treatment by way of the system 100 for TTM.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A pad for targeted temperature management (“TTM”), comprising:
a multilayered pad body including:
a conduit layer including a plurality of conduits moveably disposed in a thermally conductive medium, each conduit of the plurality of conduits configured to convey a temperature-controlled fluid as a supply fluid from a hydraulic system or convey a return fluid back to the hydraulic system;
a patient-interfacing layer over the conduit layer configured for placement on a patient’s body, the patient-interfacing layer including a thermally conductive film configured to retain the plurality of conduits and the thermally conductive medium in the conduit layer; and
an insulation layer over the conduit layer opposite the patient-interfacing layer;
a pad inlet connector configured for charging the conduit layer with the supply fluid; and
a pad outlet connector configured for discharging the return fluid from the conduit layer.

2. The pad of claim 1, wherein one or more conduits of the plurality of conduits are configured to be moved away from one or more other conduits of the plurality of conduits for selective placement of the one-or-more conduits, the one-or-more other conduits, or a combination thereof upon the patient’s body.

3. The pad of claim 1, wherein the pad inlet connector includes an inlet manifold and the pad outlet connector includes an outlet manifold, the inlet manifold configured for charging the plurality of conduits with the supply fluid, and the outlet manifold configured for discharging the return fluid from the plurality of conduits.

4. The pad of claim 3, wherein each conduit of the plurality of conduits is in parallel with other conduits of the plurality of conduits.

5. The pad of claim 1, wherein each conduit of the plurality of conduits is fluidly connected to another conduit of the plurality of conduits, thereby providing one or more fluid paths through the pad from the pad inlet connector to the pad outlet connector.

6. The pad of claim 1, wherein fluid connections between the plurality of conduits are between ends of the plurality of conduits, internal portions of the plurality of conduits, or a combination thereof, the plurality of conduits being moveable between the fluid connections.

7. The pad of claim 1, further comprising:
a plurality of thermoelectric devices disposed in the conduit layer orthogonal to the plurality of conduits, the plurality of thermoelectric devices configured to undergo a temperature change upon application of a voltage across the plurality of thermoelectric devices; and
an electrical connector configured for establishing an operable connection with a control module for controlling the plurality of thermoelectric devices.

8. A system for targeted temperature management (“TTM”), comprising:
a control module including a hydraulic system configured to provide a temperature-controlled fluid;
a primary fluid delivery line (“FDL”) configured to convey the temperature-controlled fluid from the hydraulic system as a supply fluid and convey the temperature-controlled fluid back to the hydraulic system as a return fluid; and
one or more pads configured for placement on one or more portions of a patient’s body, respectively, each pad of the one-or-more pads including:
a multilayered pad body including:
a conduit layer including a plurality of conduits moveably disposed in a thermally conductive medium, each conduit of the plurality of conduits configured to convey the temperature-controlled fluid as the supply fluid from the hydraulic system or convey the return fluid back to the hydraulic system;
a patient-interfacing layer over the conduit layer configured for placement on the patient’s body, the patient-interfacing layer including a thermally conductive film configured to retain the plurality of conduits and the thermally conductive medium in the conduit layer; and
an insulation layer over the conduit layer opposite the patient-interfacing layer;
a pad inlet connector configured for charging the conduit layer with the supply fluid; and
a pad outlet connector configured for discharging the return fluid from the conduit layer.

9. The system of claim 8, wherein one or more conduits of the plurality of conduits are configured to be moved away from one or more other conduits of the plurality of conduits for selective placement of the one-or-more conduits, the one-or-more other conduits, or a combination thereof upon the patient’s body.

10. The system of claim 8, wherein the pad inlet connector includes an inlet manifold and the pad outlet connector includes an outlet manifold, the inlet manifold configured for charging the plurality of conduits with the supply fluid, and the outlet manifold configured for discharging the return fluid from the plurality of conduits.

11. The system of claim 10, wherein each conduit of the plurality of conduits is in parallel with other conduits of the plurality of conduits.

12. The system of claim 8, wherein each conduit of the plurality of conduits is fluidly connected to another conduit of the plurality of conduits, thereby providing one or more fluid paths through the pad from the pad inlet connector to the pad outlet connector.

13. The system of claim 8, wherein fluid connections between the plurality of conduits are between ends of the plurality of conduits, internal portions of the plurality of conduits, or a combination thereof, the plurality of conduits being moveable between the fluid connections.

14. The system of claim 8, the pad body further including:
a plurality of thermoelectric devices disposed in the conduit layer orthogonal to the plurality of conduits, the plurality of thermoelectric devices configured to undergo a temperature change upon application of a voltage across the plurality of thermoelectric devices; and an electrical connector configured for establishing an operable connection with the control module for controlling the plurality of thermoelectric devices.

15. The system of claim 8, the control module further including a console including one or more processors, primary memory including read-only memory (“ROM”) and random-access memory (“RAM”), and instructions stored in the ROM configured to instantiate one or more processes in the RAM for TTM with the control module.

16. The system of claim 8, the hydraulic system comprising:
a chiller evaporator configured for fluid cooling;
a heater configured for fluid heating, the chiller evaporator and the heater, together, configured to provide the temperature-controlled fluid;
a hydraulic-system outlet configured for discharging the supply fluid from the hydraulic system; and
a hydraulic-system inlet configured for charging the hydraulic system with the return fluid to continue to produce the temperature-controlled fluid.

17. The system of claim 8, further comprising one or more secondary fluid delivery lines (“FDLs”) configured to convey the supply fluid from the hydraulic system by way of the primary FDL and convey the return fluid back to the hydraulic system by way of the primary FDL.

18. A method for targeted temperature management (“TTM”), comprising:
placing a pad on a patient’s body, the pad including:
a multilayered pad body including:
a conduit layer including a plurality of conduits moveably disposed in a thermally conductive medium, each conduit of the plurality of conduits configured to convey a temperature-controlled fluid as a supply fluid from a hydraulic system of a control module or convey a return fluid back to the hydraulic system;
a patient-interfacing layer over the conduit layer configured for placement on the patient’s body, the patient-interfacing layer including a thermally conductive film configured to retain the plurality of conduits and the thermally conductive medium in the conduit layer; and an insulation layer over the conduit layer opposite the patient-interfacing layer;
a pad inlet connector configured for charging the conduit layer with the supply fluid; and
a pad outlet connector configured for discharging the return fluid from the conduit layer;
charging the plurality of conduits of the pad with the supply fluid provided by the hydraulic system of the control module by way of a combination of fluidly connected fluid delivery lines (“FDLs”) including a primary FDL and a secondary FDL; and
circulating the supply fluid through the pad to cool or warm the patient’s body as needed in accordance with TTM.

19. The method of claim 18, wherein one or more conduits of the plurality of conduits are configured to be moved away from one or more other conduits of the plurality of conduits for selective placement of the one-or-more conduits, the one-or-more other conduits, or a combination thereof upon the patient’s body.

20. The method of claim 18, wherein the pad inlet connector includes an inlet manifold and the pad outlet connector includes an outlet manifold, the inlet manifold configured for charging the plurality of conduits with the supply fluid, and the outlet manifold configured for discharging the return fluid from the plurality of conduits.

21. The method of claim 20, wherein each conduit of the plurality of conduits is in parallel with other conduits of the plurality of conduits.

22. The method of claim 18, wherein each conduit of the plurality of conduits is fluidly connected to another conduit of the plurality of conduits, thereby providing one or more fluid paths through the pad from the pad inlet connector to the pad outlet connector.

23. The method of claim 18, wherein fluid connections between the plurality of conduits are between ends of the plurality of conduits, internal portions of the plurality of conduits, or a combination thereof, the plurality of conduits being moveable between the fluid connections.

24. The method of claim 18, the pad body further including:
a plurality of thermoelectric devices disposed in the conduit layer orthogonal to the plurality of conduits, the plurality of thermoelectric devices configured to undergo a temperature change upon application of a voltage across the plurality of thermoelectric devices; and
an electrical connector configured for establishing an operable connection with the control module for controlling the plurality of thermoelectric devices.

25. The method of claim 18, further comprising:
fluidly connecting a secondary FDL outlet connector at a pad-connecting end of the secondary FDL to the pad inlet connector; and
fluidly connecting a secondary FDL inlet connector at the pad-connecting end of the secondary FDL to the pad outlet connector.

26. The method of claim 18, wherein circulating the supply fluid through the conduit layer transfers heat between the temperature-controlled fluid and the patient’s body by thermal conduction through the conduit layer.

27. The method of claim 18, wherein circulating the supply fluid through the conduit layer includes circulating a cool fluid through the conduit layer to bring the patient into hypothermia from normothermia.

28. The method of claim 18, wherein circulating the supply fluid through the conduit layer includes circulating a warm fluid through the conduit layer to bring the patient into normothermia from hypothermia.

29. The method of claim 18, wherein circulating the supply fluid through the conduit layer includes circulating a warm fluid through the conduit layer to bring the patient into hyperthermia from normothermia.

30. The method of claim 18, wherein circulating the temperature-controlled fluid through the conduit layer includes circulating a cool fluid through the conduit layer to bring the patient into normothermia from hyperthermia.
